(19) United States
(12) Patent Application Publication
(10) Pub. No.: US 2005/0196477 A1
(43) Pub. Date: Sep. 8, 2005

(54) Mangosteen protein supplement

(76) Inventors: Aaron R. Garrity, Pleasant Grove, UT (US); Joseph C. Morton, Springville, UT (US); Paula Morrison, Union, KY (US); Greg Horn, Wyoming, OH (US)

Correspondence Address: MCANDREWS HELD & MALLOY, LTD, 500 WEST MADISON STREET, SUITE 3400, CHICAGO, IL, 60661, US

(21) Appl. No.: 10/987,447

(22) Filed: Nov. 12, 2004

Related U.S. Application Data
(60) Provisional application No. 60/519,853, filed on Nov. 13, 2003.

(51) Int. Cl. A61K 35/78

(52) U.S. Cl. 424/769; 424/777

(57) ABSTRACT

A nutraceutical protein supplement composition containing xanthones and proteins is disclosed. The protein supplement, preferably, contains xanthones derived from fruit of the Garcinia mangostana L. plant, otherwise known as the mangosteen plant, especially from the pericarp or the whole fruit of the mangosteen plant.

### RELATED APPLICATIONS

This application is a non-provisional utility patent application claiming priority to U.S. provisional patent application Ser. No. 60/519,853, filed Nov. 13, 2003, now pending. The entire specification of the provisional application referred to above are hereby incorporated by reference to provide continuity of disclosure.

### FIELD OF THE INVENTION

The presently described technology relates to a nutraceutical composition comprising xanthones and proteins. More particularly, the invention relates to a protein supplement comprising xanthones derived from, for example, fruit of the Garcinia mangostana L. plant, otherwise known as the mangosteen plant.

### BACKGROUND OF THE INVENTION

Protein supplements are well-known in the art of health and nutrition products. Proteins, one of the three main classes of food, are found in meat, eggs and beans. They are usually large, complex compounds consisting of many amino acids connected in varying sequences and forming various shapes. Upon ingestion, the body breaks down proteins and turns them into essential amino acids. Due to increasingly busy lifestyles and greater emphasis on maintaining a healthy diet, taking protein supplements has become a convenient method of maintaining or even increasing one's energy and muscle mass. Protein supplements are ideal for quickly enhancing strength and energy.

The medicinal properties of the Garcinia mangostana L. plant have increasingly been the subject of recent pharmacological and clinical studies. These studies have shown that some of the natural compounds derived from the plant yield surprising medicinal benefits, especially the xanthone compounds. The history of the Garcinia mangostana L. plant and the pharmacological benefits of individual xanthone compounds are described in more particular detail in U.S. Pat. No. 6,730,333 (Garrity et al.), which is incorporated herein by reference.

Despite the known use of protein supplements and the documented medicinal benefits of xanthones, there exists a need in the art for a protein supplement that offers the advantages of xanthone compounds. Moreover, there is a need in the art for a protein supplement comprising therapeutic amounts of xanthones. There is a further need in the art for a protein supplement that comprises xanthones derived from the fruit of the Garcinia mangostana L. plant, otherwise known as the mangosteen plant. There is an additional need in the art for a protein supplement comprising xanthones from the pericarp or a mixture of the pericarp and fruit pulp of the mangosteen plant.

### BRIEF SUMMARY OF THE INVENTION

The presently described technology relates to nutraceutical compositions containing xanthones and proteins.

In one aspect, the presently described technology provides a protein supplement that contains at least one protein and at least on xanthone. The protein supplement can also contain soluble fibers, vitamins, minerals, flavorants or a combination thereof.

Preferably, xanthones to be used in the presently described technology are derived from natural plant sources, especially from fruit of Garcinia mangostana L. plant. For example, the pericarp, fruit pulp or the whole fruit of the fruit of Garcinia mangostana L. plant can be used to provide an excellent source of xanthones.

### BRIEF DESCRIPTION OF SEVERAL VIEWS OF THE DRAWINGS

[Not Applicable]

### DETAILED DESCRIPTION OF THE INVENTION

It is understood that the terminology used herein is used for the purpose of describing particular embodiments only, and is not intended to limit the scope of the presently described technology. It is also understood that, as used herein and in the appended claims, the singular forms “a,” “an,” and “the” include plural reference, unless the context clearly dictates otherwise.

Unless defined otherwise, all technical and scientific terms used herein have the same meanings as commonly understood by one of ordinary skill in the art to which this invention belongs. While various methods, compositions, and materials of the presently described technology are described herein, any methods and materials similar or equivalent to those described herein can by used in the practice or testing of the presently described technology. All references cited herein are incorporated by reference in their entirety.

The protein in protein supplements can be derived from a number of sources well known in the art. For example, soy, whey or egg protein concentrates are an excellent source of protein. These protein concentrates are usually formed through acid extraction or filtration from the source material and dehydrated at high heats. Usually these protein concentrates contain 60-70% protein by dry weight. Protein isolates are another acceptable source of protein for the presently described technology. The protein isolates are formed through known techniques for separating fats and carbohydrates from protein. These methods include washing the protein source with water or alcohol and filtering the washed material to isolate the proteins. Protein isolates usually contain 90-95% protein by dry weight. Protein isolation is effective for whey and egg source proteins. Hydrolyzation is another common method of treating proteins. Hydrolyzed proteins may be used in the presently described technology. In this process, the large molecule proteins are broken into peptide chains, which can be absorbed more easily into the digestive tract.

To assist in digestion of the protein supplement of the presently described technology, soluble fibers may be used. Soluble fibers are fibers that can substantially undergo fermentation in the colon to produce short chain fatty acids. Examples of suitable soluble fibers include carubin, pectin, tragacanth, cereal bet-glucan and the like. The fibers may be hydrolysed or not. Artificial fiber complexes may also be used in the presently described technology. These include complexes composed of cellulose and guar gum or psyllium. Guar gum is also useful for improving handling, smoothness and shelf life of the finished product.

The presently described technology may also be fortified with vitamins and minerals, to provide additional health and energy benefits. Although there is no limit as to the type and variety of vitamins and minerals that can be employed with the presently described technology, and such knowledge is well within the realm of one of ordinary skill in the art, examples of vitamins and minerals suitable for incorporation in the compositions of the presently described technology include vitamin A, vitamin B3, vitamin B5, vitamin B6, vitamin B12, vitamin B12, vitamin C, vitamin D, vitamin E, vitamin K, folic acid, thiamin, riboflavin, niacin, biotin acceptable forms of panthotenic acid, calcium, sodium, potassium, phosphorous, magnesium, manganese, copper, zinc, iron, selenium, chromium and molybdenum.

Flavorants may also be employed in the presently described technology to provide enhanced taste and a variety of palatable product options. In a preferred embodiment of the presently described technology, chocolate in the form of cocoa powder may be used. Other flavorants, such as vanilla may also be employed. Those of ordinary skill in the art will appreciate that the type and variety of flavorants is not limited and ultimately depends on the desired final product taste.

The xanthones used in the presently described technology may be derived from a number of different sources. Preferably, the xanthones are natural xanthones derived from the pericarp, fruit pulp or the whole fruit of the fruit of the Garcinia mangostana L. plant, although other sources would also be efficacious. In one embodiment of the invention, whole mangosteen fruit of the mangosteen fruit is ground into a mixture containing pericarp and fruit pulp. Water is then removed from the mixture through standard techniques of dehydration well know in the industry, including vacuum dehydration. The resulting dehydrated mixture of natural xanthones derived from the pericarp and fruit pulp of the mangosteen fruit is an excellent source of natural, holistic xanthone compounds. In another embodiment of the presently described technology, the xanthones are extracted from a mangosteen whole fruit mixture or from the pericarp of the mangosteen fruit.

The protein supplement of the presently described technology can be prepared in any form that is suitable for oral administration. Example of oral preparations can include a tablet, powder, granule, capsule, solution, syrup, elixir, suspension, etc.

The presently described technology will now be explained with reference to the following examples, which are given for illustration only and are not intended to be limiting thereof.

### EXAMPLE 1

A protein supplement was made by combining the ingredients listed the following table:

| Ingredients | Amount |
|---|---|
| Whey Protein Isolate | 20 grams per 8 oz |
| Guar Gum | 1 gram per 8 oz |
| Vitamin B3 | 10% RDI per 8 oz |
| Vitamin B5 | 10% RDI per 8 oz |
| Vitamin B6 | 10% RDI per 8 oz |
| Vitamin B12 | 10% RDI per 8 oz |
| Cocoa powder | To desired taste |
| Dehydrated pericarp- | 0.50 oz. |
| pulp mixture of whole | |
| mangosteen | |

### EXAMPLE 2

A protein supplement was made by combining the ingredients listed the following table:

| Ingredients | Amount |
|---|---|
| Whey Protein Isolate | 20 grams per 8 oz |
| Fibersol-2 | 1 gram per 8 oz |
| Vitamin B3 | 10% RDI per 8 oz |
| Vitamin B5 | 10% RDI per 8 oz |
| Vitamin B6 | 10% RDI per 8 oz |
| Vitamin B12 | 10% RDI per 8 oz |
| Cocoa powder | To desired taste |
| Dehydrated pericarp- | 0.50 oz. |
| pulp mixture of whole | |
| mangosteen | |

The presently described technology may be embodied in other specific forms without departing from its spirit or essential characteristics. The described embodiments are to be considered in all respects only as illustrative and not restrictive. The scope of the presently described technology is, therefore, indicated by the appended claims rather than by the foregoing description. All changes that come within the meaning and range of equivalency of the claims are to be embraced within their scope.

1. A protein supplement comprising at least one protein and at least one xanthone.

2. The protein supplement of claim 1 and further comprising at least one ingredient selected from the group consisting of soluble fibers, vitamins, minerals, flavorants, and combinations thereof.

3. The protein supplement of claim 1, wherein the at least one xanthone is derived from natural plant sources.

4. The protein supplement of claim 1, wherein the at least one xanthone is derived from fruit of Garcinia mangostana L. plant.

5. The protein supplement of claim 1, wherein the at least one xanthone is derived from pericarp of fruit of Garcinia mangostana L. plant.

6. The protein supplement of claim 1, wherein the at least one xanthone is derived from a mixture of pulp and pericarp of fruit of Garcinia mangostana L. plant.

7. The protein supplement of claim 1, wherein the at least one xanthone is derived from fruit of Garcinia mangostana L. plant by a process comprising:
grinding whole fruit of Garcinia mangostana L. plant into a mixture of pericarp and fruit pulp; and
dehydrating the mixture, wherein the dehydrated mixture is used to make the protein supplement.

8. The protein supplement of claim 1, wherein the at least one xanthone is derived from fruit of Garcinia mangostana L. plant by a process comprising:
grinding whole fruit of Garcinia mangostana L. plant into a mixture of pericarp and fruit pulp; and
extracting xanthones from the mixture.

9. The protein supplement of claim 1, wherein the at least one protein is derived from protein concentrates, protein isolates, hydrolyzed proteins, or a combination thereof.

10. The protein supplement of claim 2, wherein the soluble fibers are selected from the group consisting of carubin, pectin, tragacanth, cereal bet-glucan, cellulose, guar gum, psyllium, and combinations thereof.

11. The protein supplement of claim 2, wherein the vitamins and minerals are selected from the group consisting of vitamin A, vitamin B3, vitamin B5, vitamin B6, vitamin B12, vitamin B12, vitamin C, vitamin D, vitamin E, vitamin K, folic acid, thiamin, riboflavin, niacin, biotin acceptable forms of panthotenic acid, calcium, sodium, potassium, phosphorous, magnesium, manganese, copper, zinc, iron, selenium, chromium, molybdenum, and combinations thereof.

12. The protein supplement of claim 1, wherein the protein supplement is in the form of tablet, powder, granule, capsule, solution, syrup, elixir, or suspension.
